(19) United States
(12) Patent Application Publication
(10) Pub. No.: US 2011/0177573 A1
(43) Pub. Date: Jul. 21, 2011

(54) Microbial Treatment of Lignocellulosic Biomass

(76) Inventors: Sarah All, White River Junction, VT (US); David Anthony Hogsett, Grantham, NH (US)

(21) Appl. No.: 13/055,783

(22) PCT Filed: Aug. 3, 2009
(86) PCT No.: PCT/US09/52559

§ 371 Date: Apr. 7, 2011

Related U.S. Application Data
(62) Division of application No. 61/085,435, filed on Aug. 1, 2008.

(51) Int. Cl. C12P 7/06

(52) U.S. Cl. 435/161

(57) ABSTRACT

Aspects of the present invention relate to methods of microbially treating lignocellulosic biomass using cellulose- and/or hemicellulose-degrading bacteria. In certain embodiments, the microbially treated material is then subjected to thermal and/or chemical pretreatment. In tandem with the microbial treatment the thermal and/or chemical pretreatment may result in the production of fewer degradation products, thereby allowing for higher overall yields of ethanol per ton of starting biomass.

### RELATED APPLICATIONS

This application claims the benefit of priority to U.S. Provisional Patent Application Ser. No. 61/085,435, filed Aug. 1, 2008; the contents of which are hereby incorporated by reference.

### BACKGROUND OF THE INVENTION

The production of ethanol from lignocellulosic material involves the breakdown and hydrolysis of lignocellulose-containing materials into disaccharides, such as cellobiose, and ultimately monosaccharides, such as glucose and xylose. Microbial agents, including yeasts, then convert the monosaccharides into ethanol in a fermentation reaction which can occur over several days or weeks.

Currently, lignocellulosic feedstocks are pretreated to render the lignocellulosic material more accessible to subsequent enzymatic hydrolysis. Pretreatment methods can be classified into physical, chemical, physicochemical, and biological, depending on the mode of action. A further discussion of these pretreatments can be found in Holtzapple et al. (U.S. Pat. No. 5,865,898; hereby incorporated by reference).

A fundamental objective of pre-treatment is to reduce the crystallinity of the cellulose and to dissociate the hemicellulose-cellulose-lignin complex. The digestibility of the cellulose typically increases with the degree of severity of the pre-treatment. This increase in digestibility is often directly related to the increase in the available surface area (ASA) of the cellulose materials, which facilitates the hydrolysis by enzymes, such as cellulases. That said, while some portions (e.g., cellulose) of untreated feedstock are recalcitrant, others are more amenable to degradation. Unfortunately, the conditions required to render the most recalcitrant portions of untreated feedstock available for hydrolysis also cause partial degradation of the hemicellulose fraction. In addition, toxic compounds, such as furfural and HMF, may be produced as the severity of the pretreatment increases. On the other hand, if the severity of pretreatment is reduced to prevent degradation of hemicelluloses, lower yields in downstream processes often result due to an incomplete hydrolysis of the cellulosic fraction. Traditionally, a balance is found to maximize cellulose hydrolysis while minimizing degradation of the hemicellulose. A pretreatment scheme that minimizes or eliminates the hemicellulose degradation provides the opportunity to use increased severity in chemical pretreatment, thereby increasing the yield of fermentable sugars.

Biological pretreatments can use fungi for microbial de-lignification to make cellulose more accessible. Major biological lignin degraders are the higher fungi, such as Ascomycetes and Basidiomycetes. Fungal degradation is a slow process and most fungi attack not only lignin, but also cellulose, thus resulting in a mixture of lignin fragments and sugars. Acid-producing anaerobic bacteria can also be used to increase lignocellulose digestibility. White rot and other fungi have been employed in the pulp and paper industry to reduce lignin content of lignocellulosic material and have been investigated as a form of microbial pretreatment for lignocellulosic biomass. These processes have been shown to increase the digestibility of the material following thermochemical pretreatments. However, pretreatment with aerobic fungi often results in the net loss of fermentable sugars.

It is an object of this invention to provide a method of treating lignocellulosic material with hemicellulose-degrading and/or cellulose-degrading bacteria that are capable of significantly hydrolyzing untreated material. Other objects of the invention will be apparent from the following disclosure, claims, and drawings.

### SUMMARY OF THE INVENTION

Aspects of the present invention relate to a method of microbially treating lignocellulosic material using cellulose- and/or hemicellulose-degrading bacteria. In certain embodiments, the treated material is subjected to thermal and/or chemical pretreatment. As described herein, such thermal and/or chemical pretreatment may result in fewer degradation products, and therefore allows for overall higher ethanol yields per ton of original biomass.

In another embodiment, lignocellulosic material is treated and stripped of easy to hydrolyze material. In one aspect, bacteria are added to untreated lignocellulosic material, thereby removing xylan, arabinans, or other easily degraded constituents and enabling depolymerization and fermentation of hemicellulose and cellulose.

In one embodiment, the present invention features a method of processing lignocellulosic material, wherein cellulose- and/or hemicellulose-degrading bacteria are added into a reactor containing a sample of untreated lignocellulosic material, resulting in a liquid product, and a solid product. In certain embodiments, ethanol may be produced. In certain embodiments, the solid product may be subjected to further processes, including autohydrolysis and consolidated bioprocessing. In other embodiments, the liquid product may be subjected to hydrolysate fermentation.

In yet another embodiment, the bacterium may be hemicellulose- and/or cellulose-degrading bacteria. In one embodiment, the bacteria used in the methods of the invention are thermophilic microorganisms. In another embodiment, the thermophilic bacteria are of the genera Thermoanaerobacterium or Thermoanaerobacter. In yet another embodiment, the bacteria are cellulolytic, xylanolytic thermophilic anaerobes.

In certain other embodiments, it may be desirable to perform the treatment step at various points through the overall process. In one embodiment, it may be desirable to perform the microbial process without subsequent pretreatment. In such cases, the expense and the yield losses related to pretreatment may be decreased. It will be appreciated that it may also be desirable to remove various components of the mixture, such as sugars, e.g., pentoses or hexoses, during the methods of the invention so as to minimize exposure to a subsequent pretreatment process.

In another aspect, fermentation products, such as ethanol, may enhance the effectiveness of any subsequent thermal and/or chemical treatment. Alternatively, in one embodiment, ethanol may be readily removed from the treatment mash using conventional processes.

It will be appreciated that the methods described herein will provide recovery of ethanol in high yields, reduction of feed stream to chemical pretreatment, and subsequent lower energy and chemical demands.

### BRIEF DESCRIPTION OF THE FIGURES

FIG. 1 depicts as a function of time the percent hydrolysis of glucan and xylan in corn stover by C. thermocellum at a solids loading of 0.2% glucan.

FIG. 2 depicts schematically a matrix of processes for producing ethanol from lignocellulosic material, the processing including microbial treatment, autohydrolysis pretreatment, hydrolysate fermentation, and consolidated bioprocessing.

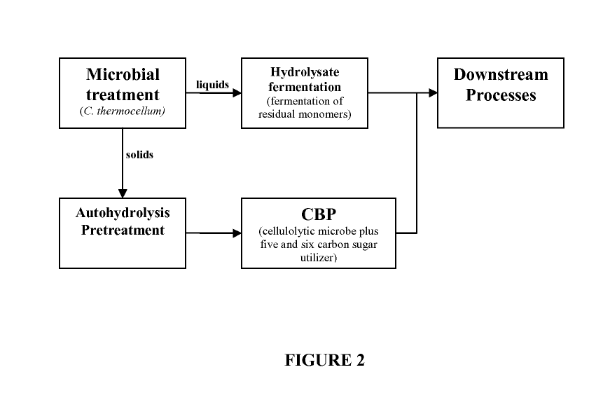

FIG. 3 tabulates the concentrations (mM) of simple sugars produced by A. thermophilum DSM 6725 after growth (90 h) on insoluble forms of poplar, switchgrass, crystalline cellulose and xylan. Concentrations were determined by gas chromatography-mass spectrometry (GC-MS). ND: not detected.

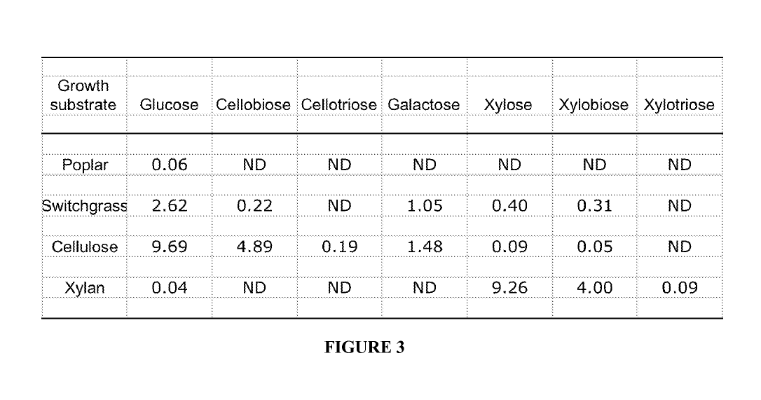

### DETAILED DESCRIPTION OF THE INVENTION

### Overview

One aspect of the present invention relates to a process by which cellulose- and/or hemicellulose-degrading bacteria are used to treat lignocellulosic material microbially. The bacteria can depolymerize and ferment hemicellulose and cellulose. The material following microbial treatment will be rich in recalcitrant material and stripped of easy to hydrolyze material, resulting in a smaller feed stream to chemical pretreatment and lower energy and chemical demands.

In certain embodiments, the stripped and microbially treated material is subjected to subsequent thermal and/or chemical pretreatment. Energy and chemical demand during the subsequent pretreatments may be reduced as a result of the microbial pretreatment. The subsequent pretreatments will produce fewer degradation products and may allow for a higher overall ethanol yield.

### Definitions

As used herein, the term “biomass” refers to a cellulose-, hemicellulose-, or lignocellulose-containing material. Biomass is commonly obtained from, for example, wood, plants, residue from agriculture or forestry, organic component of municipal and industrial wastes, primary sludges from paper manufacture, waste paper, waste wood (e.g., sawdust), agricultural residues such as corn husks, corn cobs, rice hulls, straw, bagasse, starch from corn, wheat oats, and barley, waste plant material from hard wood or beech bark, fiberboard industry waste water, bagasse pity, bagasse, molasses, post-fermentation liquor, furfural still residues, aqueous oak wood extracts, rice hull, oats residues, wood sugar slops, fir sawdust, naphtha, corncob furfural residue, cotton balls, rice, straw, soybean skin, soybean oil residue, corn husks, cotton stems, cottonseed hulls, starch, potatoes, sweet potatoes, lactose, waste wood pulping residues, sunflower seed husks, hexose sugars, pentose sugars, sucrose from sugar cane and sugar beets, corn syrup, hemp, and combinations of the above.

The terms “lignocellulosic material” and “lignocellulosic substrate” mean any type of lignocellulosic material comprising cellulose, such as but not limited to, non-woody-plant lignocellulosic material, agricultural wastes, forestry residues, paper-production sludge, waste-water-treatment sludge, corn fiber from wet and dry mill corn ethanol plants, and sugar-processing residues.

In a non-limiting example, the lignocellulosic material can include, but is not limited to, grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, wheat straw, canola straw, oat straw, oat hulls, and corn fiber; stover, such as but not limited to soybean stover, and corn stover, or any combination thereof.

Lignocellulosic materials are composed of mainly cellulose, hemicellulose, and lignin. Generally, a lignocellulosic material, on a dry basis, may contain about 50% (w/w) cellulose, about 30% (w/w) hemicellulose, and about 20% (w/w) lignin. The lignocellulosic material can be of lower cellulose content, for example, at least about 20% (w/w), 30% (w/w), 35% (w/w), or 40% (w/w).

The term “reactor” may mean any vessel suitable for practicing a method of the present invention. The dimensions of the pretreatment reactor may be sufficient to accommodate the lignocellulose material conveyed into and out of the reactor, as well as additional headspace around the material. In a non-limiting example, the headspace may extend about one foot around the space occupied by the materials. Furthermore, the pretreatment reactor may be constructed of a material capable of withstanding the pretreatment conditions. Specifically, the construction of the reactor should be such that the pH, temperature and pressure do not affect the integrity of the vessel.

The size range of the substrate material varies widely and depends upon the type of substrate material used as well as the requirements and needs of a given process. In a preferred embodiment of the invention, the lignocellulosic raw material may be prepared in such a way as to permit ease of handling in conveyors, hoppers and the like. In the case of wood, the chips obtained from commercial chippers may be suitable; in the case of straw it may be desirable to chop the stalks into uniform pieces about 1 to about 3 inches in length. Depending on the intended degree of pretreatment, the size of the substrate particles prior to pretreatment may range from less than a millimeter to inches in length. The particles need only be of a size that is reactive.

### Microbial Treatments

One aspect of microbial treatment involves adding hemicellulose-degrading and/or cellulose-degrading bacteria to lignocellulosic material. In certain embodiments, the treated material is subjected to thermal and/or chemical pretreatment.

By adding the bacteria to untreated lignocellulosic biomass, xylan, arabinans, or other easily degraded constituents are often easily removed resulting in depolymerization and fermentation of hemicellulose. Some of the advantages of the microbial treatment include (1) reduced amount of HMF (Hydroxymethylfurfural) and furfural generated and (2) reduced amount of lost hemicellulose sugars, thereby increasing the yield of fermentable sugars. HMF and furfural can interfere with the enzymatic reactions during fermentation; therefore, reduced concentrations of such inhibitory compounds will increase the yield efficiency of the process. Furthermore, by removing xylan, arabinans, and other easily degraded constituents, a microbial treatment that would be capable of depolymerizing and fermenting portions of the hemicellulose would reduce the amount of hemicellulose sugars lost during subsequent thermal and/or chemical pretreatment of the lignocellulosic biomass.

Lignocellulosic materials require treatment to increase the accessibility of hemicellulose, cellulose, and other components for further processing. In certain embodiments, further processing includes enzymatic hydrolysis.

The native structure of lignocellulosics inhibits degradation. In addition to the highly-resistant crystalline structure of cellulose, the lignin surrounding the cellulose forms a physical barrier. Accordingly, the sites available for attack (e.g., by enzymes) are limited. One idealized outcome of treatment, therefore, would be to reduce lignin content with a concomitant reduction in crystallinity and increase in surface area.

In certain embodiments, the lignocellulosic materials may be soaked in water or other suitable liquid(s) prior to processing or treatment. In certain embodiments, the excess water may be drained off the lignocellulosic materials. In certain embodiments, the soaking may be done prior to conveying into a reactor, or subsequent to entry (i.e., inside a pretreatment reactor).

The size range of the substrate material varies widely and depends upon the type of substrate material used as well as the requirements and needs of a given process. In certain embodiments, the lignocellulosic raw material may be prepared in such a way as to permit ease of handling with conveyors, hoppers and the like. In the case of wood, the chips obtained from commercial chippers are suitable; in the case of straw it is sometimes desirable to chop the stalks into uniform pieces about 1 to about 3 inches in length. In certain embodiments, depending on the intended degree of treatment, the size of the substrate particles prior to treatment may range from less than a millimeter to inches in length.

In certain embodiments, ultrasound treatments may be applied to processes of the present invention. See U.S. Pat. No. 6,333,181, which is hereby incorporated by reference.

### Microorganisms

The present invention features use of cellulolytic microorganisms in the methods described herein. Several microorganisms determined from literature to be cellulolytic have been characterized by their ability to grow on microcrystalline cellulose as well as a variety of sugars. In a non-limiting example, cellulolytic microorganisms may include Clostridium thermocellum, Clostridium cellulolyticum, Thermoanaerobacterium saccharolyticum, C. stercorarium, C. stercorarium II, Caldiscellulosiruptor kristjanssonii, and C. phytofermentans.

Several microorganisms determined from literature to be both cellulolytic and xylanolytic have been characterized by their ability to grow on microcrystalline cellulose and birchwood xylan as well as a variety of sugars. Cellulolytic and xylanolytic microorganism may be used in the present invention, including, but not limited to, Clostridium cellulolyticum, Clostridium stercorarium subs. leptospartum, Caldicellulosiruptor kristjanssonii and Clostridium phytofermentans.

Strains of Anaerocellum thermophilum and Caldicellulosiruptor saccharolyticus have also proven to be cellulolytic, xylanolytic thermophilic anaerobes. For example, A. thermophilum strain DSM 6725 is able to utilize efficiently as carbon and energy sources untreated forms of both low-lignin (napier and bermuda) and high-lignin (switchgrass) grasses and a hardwood (poplar); cell densities of >10⁸ cells/mL were obtained in 20 h. The cellulose-degrading ability of A. thermophilum DSM 6725 is comparable to that of cellulolytic C. thermocellum, but A. thermophilum DSM 6725 has the advantage of a higher optimum growth temperature (75° C. in comparison to 60° C.). The former organism also proved to grow equally well on both soluble and insoluble biomass. C. saccharolyticus DSM 8903 also degrades crystalline cellulose and is able to grow at 70° C. on insoluble switchgrass in a manner similar to that of A. thermophilum DSM6725. In certain embodiments, hydrogen, acetate, or lactate may be formed in the microbial pretreatment of biomass according to the methods of present invention. In certain embodiments, glucose, cellobiose, xylose, or xylobiose may be formed in the microbial pretreatment of biomass according to the methods of the present invention.

In a non-limiting example, the cellulose-degrading bacteria can include Clostridium termitidis, Micromonospora propionici and Clostridium sp., Streptomyces sp. and Micromonospora sp., Staphylococcus saprophyticus, Micrococcus luteus and Mc. roseus, Mm acetiformici, and Arthrobacter sp., Ruminococcus albus.

In a non-limiting example, the hemicellulose-degrading bacteria can include Butyrivibrio sp., Clostridium sp., and Bacteroides sp.

In certain embodiments, microbes used in ethanol fermentation, such as yeast, fungi, and Zymomonas mobilis, may also be used in the methods of microbial pretreatment of lignocellulosic material.

Aspects of the present invention also relate to the use of thermophilic microorganisms. By “thermophilic” is meant an organism that thrives at a temperature of about 45° C. or higher. Their potential in process applications in biotechnology stems from their ability to grow at relatively high temperatures with attendant high metabolic rates, production of physically and chemically stable enzymes, and elevated yields of end products. Major groups of thermophilic bacteria include eubacteria and archaebacteria. Thermophilic eubacteria include: phototropic bacteria, such as cyanobacteria, purple bacteria, and green bacteria; Gram-positive bacteria, such as Bacillus, Clostridium, Lactic acid bacteria, and Actinomyces; and other eubacteria, such as Thiobacillus, Spirochete, Desulfotomaculum, Gram-negative aerobes, Gram-negative anaerobes, and Thermotoga. Within archaebacteria are considered Methanogens, extreme thermophiles (an art-recognized term), and Thermoplasma. In certain embodiments, the present invention relates to Gram-negative organotrophic thermophiles of the genera Thermus, Gram-positive eubacteria, such as genera Clostridium, and also which comprise both rods and cocci, genera in group of eubacteria, such as Thermosipho and Thermotoga, genera of Archaebacteria, such as Thermococcus, Thermoproteus (rod-shaped), Thermofilum (rod-shaped), Pyrodictium, Acidianus, Sulfolobus, Pyrobaculum, Pyrococcus, Thermodiscus, Staphylothermus, Desulfurococcus, Archaeoglobus, and Methanopyrus. Some examples of thermophilic (including bacteria, procaryotic microorganism, and fungi), which may be suitable for the present invention include, but are not limited to: Clostridium thermosulfurogenes, Clostridium cellulolyticum, Clostridium thermocellum, Clostridium thermohydrosulfuricum, Clostridium thermoaceticum, Clostridium thermosaccharolyticum, Clostridium tartarivorum, Clostridium thermocellulaseum, Thermoanaerobacterium thermosaccarolyticum, Thermoanaerobacterium saccharolyticum, Thermobacteroides acetoethylicus, Thermoanaerobium brockii, Methanobacterium thermoautotrophicum, Pyrodictium occultum, Thermoproteus neutrophilus, Thermofilum librum, Thermothrix thioparus, Desulfovibrio thermophilus, Thermoplasma acidophilum, Hydrogenomonas thermophilus, Thermomicrobium roseum, Thermus flavas, Thermus ruber, Pyrococcus furiosus, Thermus aquaticus, Thermus thermophilus, Chloroflexus aurantiacus, Thermococcus litoralis, Pyrodictium abyssi, Bacillus stearothermophilus, Cyanidium caldarium, Mastigocladus laminosus, Chlamydothrix calidissima, Chlamydothrix penicillata, Thiothrix carnea, Phormidium tenuissimum, Phormidium geysericola, Phormidium subterraneum, Phormidium bijahensi, Oscillatoria filiformis, Synechococcus lividus, Chloroflexus aurantiacus, Pyrodictium brockii, Thiobacillus thiooxidans, Sulfolobus acidocaldarius, Thiobacillus thermophilica, Bacillus stearothermophilus, Cercosulcifer hamathensis, Vahlkampfia reichi, Cyclidium citrullus, Dactylaria gallopava, Synechococcus lividus, Synechococcus elongatus, Synechococcus minervae, Synechocystis aquatilus, Aphanocapsa thermalis, Oscillatoria terebriformis, Oscillatoria amphibia, Oscillatoria germinate, Oscillatoria okenii, Phormidium laminosum, Phormidium parparasiens, Symploca thermalis, Bacillus acidocaldarias, Bacillus coagulans, Bacillus thermocatenalatus, Bacillus licheniformis, Bacillus pamilas, Bacillus macerans, Bacillus circulans, Bacillus laterosporus, Bacillus brevis, Bacillus subtilis, Bacillus sphaericus, Desulfotomaculum nigrificans, Streptococcus thermophilus, Lactobacillus thermophilus, Lactobacillus bulgaricus, Bifidobacterium thermophilum, Streptomyces fragmentosporus, Streptomyces thermonitrificans, Streptomyces thermovulgaris, Pseudonocardia thermophila, Thermoactinomyces vulgaris, Thermoactinomyces sacchari, Thermoactinomyces candidas, Thermomonospora curvata, Thermomonospora viridis, Thermomonospora citrina, Microbispora thermodiastatica, Microbispora aerata, Microbispora bispora, Actinobifida dichotomica, Actinobifida chromogens, Micropolyspora caesia, Micropolyspora faeni, Micropolyspora cectivugida, Micropolyspora cabrobrunea, Micropolyspora thermovirida, Micropolyspora viridinigra, Methanobacterium thermoautothropicum, Anaerocellum thermophilum, Caldicellulosiruptor saccharolyticus, variants thereof, and/or progeny thereof.

In certain embodiments, the present invention relates to the use of thermophilic bacteria selected from the group consisting of Fervidobacterium gondwanense, Clostridium thermolacticum, Moorella sp., and Rhodothermus marinus.

In certain embodiments, the present invention relates to the use of thermophilic bacteria of the genera Thermoanaerobacterium or Thermoanaerobacter, including, but not limited to, species selected from the group consisting of Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brockii, variants thereof, and progeny thereof.

In certain embodiments, the present invention relates to the use of microorganisms of the genera Geobacillus, Saccharococcus, Paenibacillus, Bacillus, and Anoxybacillus, including, but not limited to, species selected from the group consisting of: Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, variants thereof, and progeny thereof.

In certain embodiments, lignocellulosic material subjected to microbial treatment outputs a mixture containing both solids and liquids, either or both of which may be subjected to further pretreatment. Solids are separated from the liquids and the solids may be subjected to subsequent autohydrolysis to dissociate the hemicellulose-cellulose-lignin complex.

In certain embodiments, autohydrolysis pretreatment may include a steam hydrolysis where lignocellulosic material is subjected to steam pressure of between 100 psig and 700 psig. A vacuum may be pulled within the reactor to remove air, for example, at a pressure of about 50 to about 300 mbar. Steam may be added to the reactor containing the lignocellulosic material at a saturated steam pressure of between about 100 psig and about 700 psig, or any amount there between; for example, the saturated steam pressure may be about 100, 150, 200, 250, 300, 350, 400, 450, 500, 550, 600, 650, or 700 psig. More preferably, a saturated steam pressure from about 140 psig to about 300 psig may be used. When no other chemical is added to the steam during the pretreatment process, unwanted by-products and/or waste material produced in some of the conventional methods are eliminated.

Nevertheless, in certain embodiments, it may be desirable to add a catalyst during or before the non-microbial pretreatment process. If an acid catalyst is used in a method of the present invention it may be of any suitable acid known in the art; for example, the acid may be sulfuric acid, sulfurous acid, and/or sulfur dioxide, or a combination thereof. The amount of acid added may be any amount sufficient to provide a pretreatment of the lignocellulosic material at the chosen pretreatment temperature. For example, the acid loading may be about 0% to about 12% by weight of the materials, or any amount therebetween; for example, the acid may be loaded at about 0, 1, 2, 3, 4, 5, 6, 7, 8, 9, 10, 11, or 12% by weight of the lignocellulosic materials. In a non-limiting example, the acid is sulfur dioxide, and it is added to the lignocellulosic material by injecting the acid as a vapor to a concentration of about 0.5% to about 4.0% the weight of lignocellulosic material.

The acid and steam may be added in any order that is suitable to the present invention. For example, the acid may be added prior to, simultaneously with, or after the addition or injection of steam into the pre-treatment reactor.

Following autohydrolysis pretreatment, pretreated lignocellulosic material may be further subjected to a process configuration known as consolidated bioprocessing (CBP). A CBP processes four biologically mediated transformations in a single step: (1) production of saccharolytic enzymes; (2) hydrolysis of carbohydrate components present in pretreated lignocellulosic material to sugars; (3) fermentation of hexose sugars; and (4) fermentation of pentose sugars. CBP offers the potential for lower cost and higher efficiency than processes involving dedicated cellulose production.

In certain embodiments, the CBP process may include cellulolytic microbes with five and six carbon sugar utilizers.

The liquid portion of the output containing residual monomers can be subjected to hydrolysate fermentation to produce ethanol. For example, yeast or Zymomonas mobilis may be used during the fermentation process.

The sugars released during pretreatment can be utilized by C. thermocellum in the case of hexoses to produce ethanol. In conjunction with an organism capable of converting pentose sugars to ethanol, additional ethanol could be produced during microbial pretreatment. In the absence of such pentosan-metabolizing organisms, the liberating pentose sugars and soluble oligomers could be removed from the microbially pretreated material by washing the remaining insoluble lignocellulose with water and collecting the pentosans.

In certain embodiments, it may be desirable to perform the microbial process without subsequent pretreatment. In such cases, the expense and the yield losses related to pretreatment may be decreased.

Fermentation products, such as ethanol, may enhance the effectiveness of any subsequent thermal and/or chemical pretreatment. Alternatively, the ethanol could be readily removed from the pretreatment mash using conventional processes.

### Exemplary Embodiments

In certain embodiments, the invention relates to a method of processing lignocellulosic material, comprising the steps of: combining in a reactor a sample of lignocellulosic material and a cellulose-degrading or a hemicellulose-degrading bacterium, resulting in a mixture; and maintaining said mixture at a temperature for a period of time, resulting in a liquid product, and a solid product.

In certain embodiments, the invention relates to a method of processing lignocellulosic material, comprising the steps of: placing a sample of lignocellulosic material in a reactor; adding to said reactor a cellulose-degrading or a hemicellulose-degrading bacterium, resulting in a mixture; and maintaining said mixture at a temperature for a period of time, resulting in a liquid product, and a solid product.

In certain embodiments, the invention relates to any one of the aforementioned methods, wherein said liquid product comprises ethanol.

In certain embodiments, the invention relates to any one of the aforementioned methods, wherein said lignocellulosic material contains, on a dry basis, at least about 20% (w/w) cellulose, at least about 10% (w/w) hemicellulose, and at least about 10% (w/w) lignin.

In certain embodiments, the invention relates to any one of the aforementioned methods, wherein said lignocellulosic material is selected from the group consisting of grass, switch grass, cord grass, rye grass, reed canary grass, miscanthus, sugar-processing residues, sugar cane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, sawdust, hardwood, and softwood.

In certain embodiments, the invention relates to any one of the aforementioned methods, wherein said lignocellulosic material is stover.

In certain embodiments, the invention relates to any one of the aforementioned methods, wherein said lignocellulosic material is corn stover or soybean stover.

In certain embodiments, the invention relates to any one of the aforementioned methods, wherein said lignocellulosic material is hardwood; and said hardwood is selected from the group consisting of willow, maple, oak, walnut, eucalyptus, elm, birch, buckeye, beech, and ash.

In certain embodiments, the invention relates to any one of the aforementioned methods, wherein said lignocellulosic material is hardwood, and said hardwood is willow.

In certain embodiments, the invention relates to any one of the aforementioned methods, wherein said lignocellulosic material is softwood; and said softwood is selected from the group consisting of southern yellow pine, fir, cedar, cypress, hemlock, larch, pine, and spruce.

In certain embodiments, the invention relates to any one of the aforementioned methods, wherein said lignocellulosic material is softwood, and said softwood is southern yellow pine.

In certain embodiments, the invention relates to any one of the aforementioned methods, wherein said cellulose-degrading bacteria is selected from the group consisting of Clostridium termitidis, Micromonospora propionici, Clostridium sp., Streptomyces sp., Micromonospora sp., Staphylococcus saprophyticus, Micrococcus luteus, Mc. roseus, Mm acetiformici, Arthrobacter sp., and Ruminococcus albus.

In certain embodiments, the invention relates to any one of the aforementioned methods, wherein said hemicellulose-degrading bacteria is selected from the group consisting of Butyrivibrio sp., Clostridium sp., and Bacteroides sp.

In certain embodiments, the invention relates to any one of the aforementioned methods, wherein said hemicellulose-degrading bacteria is Clostridium thermocellum.

In certain embodiments, the invention relates to any one of the aforementioned methods, further comprising adding a second hemicellulose-degrading or cellulose-degrading bacterium to said reactor.

In certain embodiments, the invention relates to any one of the aforementioned methods, further comprising adding a second hemicellulose-degrading or cellulose-degrading bacterium to said reactor.

In certain embodiments, the invention relates to any one of the aforementioned methods, wherein the bacterium is a thermophilic microorganism.

In certain embodiments, the invention relates to any one of the aforementioned methods, wherein the thermophilic microorganism is a bacterium selected from the group consisting of: Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brocki, Clostridium thermocellum, Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, Anaerocellum thermophilum, and Caldicellulosiruptor saccharolyticus.

In certain embodiments, the invention relates to any one of the aforementioned methods, wherein the bacterium is a cellulolytic and xylanolytic microorganism.

In certain embodiments, the invention relates to any one of the aforementioned methods, wherein the cellulolytic and xylanolytic microorganism is a bacterium selected from the group consisting of: Clostridium cellulolyticum, Clostridium stercorarium subs. leptospartum, Caldicellulosiruptor kristjanssonii, Clostridium phytofermentans, Anaerocellum thermophilum, and Caldicellulosiruptor saccharolyticus.

In certain embodiments, the invention relates to any one of the aforementioned methods, wherein said temperature is between about 50° C. and about 100° C.

In certain embodiments, the invention relates to any one of the aforementioned methods, wherein said temperature is between about 60° C. and about 90° C.

In certain embodiments, the invention relates to any one of the aforementioned methods, wherein said temperature is between about 70° C. and about 80° C.

In certain embodiments, the invention relates to any one of the aforementioned methods, wherein said temperature is between about 50° C. and about 75° C.

In certain embodiments, the invention relates to any one of the aforementioned methods, wherein said temperature is between about 50° C. and about 65° C.

In certain embodiments, the invention relates to any one of the aforementioned methods, wherein said temperature is about 75° C.

In certain embodiments, the invention relates to any one of the aforementioned methods, wherein said period of time is between about 1 day and about 11 days.

In certain embodiments, the invention relates to any one of the aforementioned methods, wherein said period of time is between about 3 days and about 9 days.

In certain embodiments, the invention relates to any one of the aforementioned methods, wherein said period of time is less than about 1 day.

In certain embodiments, the invention relates to any one of the aforementioned methods, further comprising the step of subjecting said liquid product to hydrolysate fermentation.

In certain embodiments, the invention relates to any one of the aforementioned methods, further comprising the step of subjecting said solid product to autohydrolysis pretreatment.

In certain embodiments, the invention relates to any one of the aforementioned methods, wherein the autohydrolysis pretreatment is steam hydrolysis.

In certain embodiments, the invention relates to any one of the aforementioned methods, wherein the autohydrolysis pretreatment is acid hydrolysis.

In certain embodiments, the invention relates to any one of the aforementioned methods, further comprising the step of subjecting said solid product to consolidated bioprocessing.

### EXEMPLIFICATION

The invention will be more readily understood by reference to the following examples, which are included merely for purposes of illustration of certain aspects and embodiments of the present invention, and are not intended to limit the invention.

### Example 1

### Treatment of Lignocellulosic Biomass Using Clostridium thermocellum

Preliminary experiments were performed in 125 mL batch serum bottles at 58° C., 175 rpm, under nitrogen in buffered MTC media. Kramer corn stover was loaded at 0.2% glucan. A 5% inoculum of C. thermocellum was added to bottles after sterilization and total solids were analyzed at 12, 24, 48, 72, 120, 168, and 216 hours after inoculation by quantitative saccharification for remaining glucan and xylan.

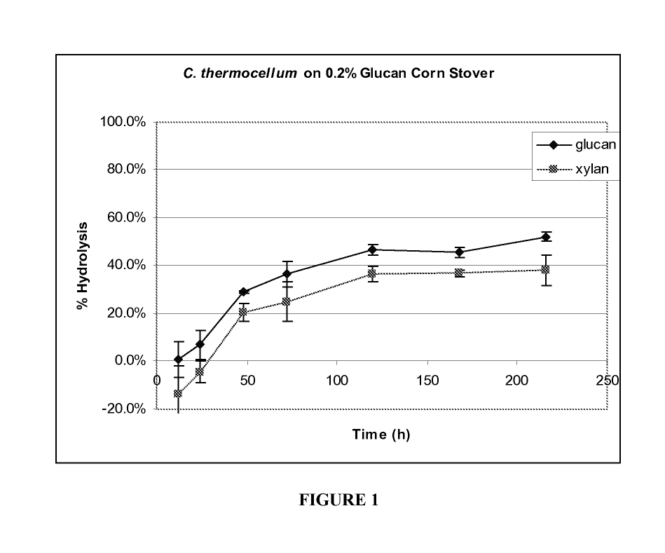

FIG. 1 depicts percent hydrolysis of glucan and xylan in corn stover by C. thermocellum at a solids loading of 0.2% glucan (˜0.6% solids) analyzed at 12, 24, 48, 72, 120, 168, and 216 hours after inoculation. Conversions are based on an initial composition of 34.4% glucan and 20% xylan. These results show that approximately 50% of the glucan and 40% of the xylan were hydrolyzed within five days. Wheat stover, switchgrass and hardwood chips may be tested. Follow up experiments may include pH controlled high solids fermentation of untreated substrates and subsequent chemical pretreatment of spent solids.

Additional experiments have shown that fermentable carbohydrates released by Clostridium thermocellum can be utilized by other organisms such as T. Saccharolyticum to produce ethanol.

### Example 2

### Treatment of Hardwood Flour with C. thermocellum

Preliminary results in the treatment of hardwood flour with C. thermocellum showed only partial conversion (less than about 5-10%) of cellulose or hemicellulose to either soluble sugars or ethanol. In certain embodiments, the microbial pretreatment may be more effective with less woody biomass, such as stover.

### Example 3

### Growth of C. thermocellum on Substrates Through Serial Transfer (Prophetic)

C. thermocellum may adapt to grow better on substrates through serial transfer. This has been shown on AFEX pretreated corn stover and may be valuable on untreated substrates as well. Without being limited by theory, serial transfer on in-house feedstocks may ensue, if hydrolysis of untreated substrates translates from corn stover to other feedstocks.

### INCORPORATION BY REFERENCE

All of the U.S. patents and U.S. published patent applications cited herein are hereby incorporated by reference. In addition, U.S. Pat. No. 4,136,207 is hereby incorporated by reference; U.S. Pat. No. 4,427,453 is hereby incorporated by reference; U.S. Pat. No. 4,600,590 is hereby incorporated by reference; U.S. Pat. No. 5,037,663 is hereby incorporated by reference; U.S. Pat. No. 5,171,592 is hereby incorporated by reference; U.S. Pat. No. 5,473,061 is hereby incorporated by reference; U.S. Pat. No. 5,865,898 is hereby incorporated by reference; U.S. Pat. No. 5,939,544 is hereby incorporated by reference; U.S. Pat. No. 6,106,888 is hereby incorporated by reference; U.S. Pat. No. 6,176,176 is hereby incorporated by reference; U.S. Pat. No. 6,348,590 is hereby incorporated by reference; U.S. Pat. No. 6,392,035 is hereby incorporated by reference; U.S. Pat. No. 6,416,621 is hereby incorporated by reference; U.S. Pat. No. 7,109,005 is hereby incorporated by reference; U.S. Pat. No. 7,198,925 is hereby incorporated by reference; U.S. published patent application 2005/0065336 is hereby incorporated by reference; and U.S. published patent application 2006/0024801 is hereby incorporated by reference; and U.S. published patent application 2007/0031953 is hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of processing lignocellulosic material, comprising the steps of: combining in a reactor a sample of lignocellulosic material and a cellulose-degrading or a hemicellulose-degrading bacterium, resulting in a mixture; and maintaining said mixture at a temperature for a period of time, resulting in a liquid product, and a solid product.

2. The method of claim 1, wherein said liquid product comprises ethanol.

3. The method of claim 1, wherein said lignocellulosic material contains, on a dry basis, at least about 20% (w/w) cellulose, at least about 10% (w/w) hemicellulose, and at least about 10% (w/w) lignin.

4. The method of claim 1, wherein said lignocellulosic material is selected from the group consisting of grass, switch grass, cord grass, rye grass, reed canary grass, miscanthus, sugar-processing residues, sugar cane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, sawdust, hardwood, and softwood.

5. The method of claim 1, wherein said lignocellulosic material is hardwood; and said hardwood is selected from the group consisting of willow, maple, oak, walnut, eucalyptus, elm, birch, buckeye, beech, and ash.

6. The method of claim 1, wherein said lignocellulosic material is hardwood, and said hardwood is willow.

7. The method of claim 1, wherein said lignocellulosic material is softwood; and said softwood is selected from the group consisting of southern yellow pine, fir, cedar, cypress, hemlock, larch, pine, and spruce.

8. The method of claim 1, wherein said lignocellulosic material is softwood, and said softwood is southern yellow pine.

9. The method of claim 1, wherein said bacterium is a cellulose-degrading bacterium selected from the group consisting of Clostridium termitidis, Micromonospora propionici, Clostridium sp., Streptomyces sp., Micromonospora sp., Staphylococcus saprophyticus, Micrococcus luteus, Mc. roseus, Mm acetiformici, Arthrobacter sp., and Ruminococcus albus.

10. The method of claim 1, wherein said bacterium is a hemicellulose-degrading bacterium selected from the group consisting of Butyrivibrio sp., Clostridium sp., and Bacteroides sp.

11. The method of claim 1, wherein said bacterium is Clostridium thermocellum.

12. (canceled)

13. (canceled)

14. The method of claim 1, wherein the bacterium is a thermophilic microorganism selected from the group consisting of Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brocki, Clostridium thermocellum, Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, Anaerocellum thermophilum, and Caldicellulosiruptor saccharolyticus.

15. (canceled)

16. The method of claim 1, wherein the bacterium is a cellulolytic and xylanolytic microorganism selected from the group consisting of Clostridium cellulolyticum, Clostridium stercorarium subs. leptospartum, Caldicellulosiruptor kristjanssonii, Clostridium phytofermentans, Anaerocellum thermophilum, and Caldicellulosiruptor saccharolyticus.

17. (canceled)

18. The method of claim 1, wherein said temperature is between about 50° C. and about 75° C.

19. (canceled)

20. The method of claim 1, wherein said period of time is between about 1 day and about 11 days.

21. (canceled)

22. The method of claim 1, wherein said period of time is less than about 1 day.

23. The method of claim 1, further comprising the step of subjecting said liquid product to hydrolysate fermentation.

24. The method of claim 1, further comprising the step of subjecting said solid product to autohydrolysis pretreatment.

25. The method of claim 24, wherein the autohydrolysis pretreatment is steam hydrolysis or acid hydrolysis.

26. (canceled)

27. The method of claim 1, further comprising the step of subjecting said solid product to consolidated bioprocessing.
